(19) United States
(12) Patent Application Publication
(10) Pub. No.: US 2010/0030083 A1
(43) Pub. Date: Feb. 4, 2010

(54) ASSESSMENT OF TISSUE RESPONSE TO STRESS

(75) Inventors: Joan E. Sanders, Sammamish, WA (US); Elizabeth A. Sorenson, Ramona, CA (US); Joshua H. Levine, Arcadia, CA (US); John R. Fergason, San Antonio, TX (US); Gregory S. Lee, Richardson, TX (US); Santosh Zachariah, Seattle, WA (US); Yak-Nam Wang, Seattle, WA (US); Paul Yager, Seattle, WA (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 12/510,115

(22) Filed: Jul. 27, 2009

Related U.S. Application Data
(60) Provisional application No. 61/084,197, filed on Jul. 28, 2008.

(51) Int. Cl. A61B 6/00

(52) U.S. Cl. 600/474

(57) ABSTRACT

An approach is described for identifying sites of imminent skin breakdown in amputee prosthesis users. Thermal recovery time (TRT) for a limb is optically determined using an infrared camera. TRT is the time interval for the temperature of the skin to achieve 70% of its maximum value during a 10-minute recovery period after a subject has completed a standing/walk-in-place procedure. A limb tolerance map is produced in which 5×5 pixel squares are colored to indicate their TRT and labeled to indicate a temperature vs. time curve (indicative of blood flow characteristics) for the square. TRT data can also be used for prosthetic fitting and socket replacement, by locating tolerant/intolerant regions on a limb and providing a visual “limb tolerance map” for a proposed socket design and applied to other areas, such as the design of shoes for patients with insensate feet, cushions for wheelchair users, and mattresses for bedridden patients.

### RELATED APPLICATIONS

This application is based on a prior copending provisional application Ser. No. 61/084,197, filed on Jul. 28, 2008, the benefit of the filing date of which is hereby claimed under 35 U.S.C. §119(e).

### GOVERNMENT RIGHTS

This invention was made with government support under grant number R01 EB000998 awarded by the National Institutes of Health (NIH)—Federal Reporting. The government has certain rights in the invention.

### BACKGROUND

There are approximately 84,500 to 114,000 new lower-limb amputations each year in the U.S. Amputation rates are rising each year, in part because of the rapid increase in diabetes incidence and also because of improvements in treatments for traumatic injury and vascular disease. More of the patients experiencing injury or vascular disease live longer, but require a limb amputation to survive. Further, the recent wars in Iraq and Afghanistan have caused an increase in the number of servicemen and women with amputation, typically young individuals who are otherwise healthy.

The incidence of skin breakdown in lower-limb amputees ranges from about 24% to 41%, and skin problems are most common in transtibial amputees of all lower-extremity amputations. Thus, skin breakdown is a significant problem in the ever-increasing transtibial amputee population. Tissue breakdown precludes the prosthesis from being used until the damaged tissue heals, a situation that is frustrating and debilitating to a person with an amputation. Severe skin breakdown may require surgical repair and possibly, further amputation to a more proximal anatomical level on the limb. The cost of a typical residual limb breakdown event has not been reported, although acute and post acute medical care costs associated with caring for persons with dysvascular amputation reportedly exceed $4.3 billion annually.

An important source of skin breakdown is a poorly fitting prosthetic socket. To be effective, a socket must be designed to transfer load to residual limb areas that can well tolerate stress. Low tolerant sites should receive less stress. Because the residual limb changes shape and stiffness over time, an amputee will often need a new prosthetic socket that better fits the changing residual limb. Specifically, for the first three years after amputation, a new prosthesis is needed more than once a year. Thus, fitting a socket to the amputee's residual limb is a not a one-time event, but instead, should be viewed as a continuing repeated process.

During prosthetic fitting or replacement, it would be useful to assess soft tissue tolerance. Prosthetic training manuals provide general guidelines for identifying typical tolerant areas. However, these guidelines only provide generalities, and not steadfast rules. The clinician must customize each socket design, relying heavily on experience combined with clinical inspection and palpation of the individual's residual limb. Although transcutaneous oxygen tension (referred to as “TcPO2”), Laser Doppler flowmetry, Doppler ultrasound, and other methods can assess limb soft tissues, data from these instruments are not necessarily indicative of load tolerance. Part of the problem is that, for the application of interest here, these instruments lack sufficient sensitivity. Exacerbating the problem, a current shortage of prosthetists in the industry is expected to increase in the future, and quality prosthetic experience is in high demand. Even experienced prosthetists face challenges in prosthetic fitting, not only because each residual limb is different, but also because palpation and inspection are not very sensitive analysis tools. It is virtually impossible to consistently make accurate predictions regarding a patient's skin tolerance. As a result, the initial fitting or socket replacement process is a trial and error procedure, and in many cases, skin irritation or breakdown occurs.

Accordingly, there is a need to develop a non-invasive imaging tool to identify sites of imminent soft tissue breakdown in prosthesis users, early on, and well before injury is clinically apparent.

One approach that might be used to solve this problem is assessment of the thermal recovery time of tissue. In bedsore tests, the skin of newly-admitted nursing home patients was subjected to a static pressure of 50 kPa for 10 minutes at locations susceptible to pressure ulcer formation. After the load was released, the time for the temperature difference between the stressed site and a site 10 to 15 cm away to reach either a maximum or a constant value was assessed. A very simple measurement instrument, a thermocouple, was used, and only data at the test and control points were collected. Results demonstrated that the recovery time correlated strongly with the risk to develop pressure ulcers, with the risk defined using data on ulceration occurrence over a one-month follow up period for the 109-person subject population (divided into eight groups) that was studied (see exemplary data 40 in the graph of FIG. 2). The method was also used to demonstrate that diabetic patients with autonomic neuropathy had an impaired recovery time after pressure relief compared with normal control subjects. These tests substantiate that thermal recovery time is a good assessment parameter for skin breakdown prediction.

Some researchers have found a different feature, temperature magnitude change, to be useful for predicting ulceration in patients with Charcot's arthropathy, neuropathy, and leprosy. However, the issue of false positives was not well-addressed. Further, it is possible that there is a weak link between temperature magnitude and skin breakdown on patients that do not have these disorders.

It would also be desirable to enhance the utility of a system or tool that can assess the breakdown of tissue for application to prosthetic fitting socket replacement, and component selection. In addition to identifying imminent breakdown sites, the system should be able to predict, for example, if a proposed socket design is going to traumatize residual limb tissues. In other words, the tool should provide a “limb tolerance map,” indicating where, in terms of skin tolerance, a prosthetist's new socket design is unacceptable. In addition, the occurrence of false positives should be reduced.

To obtain a limb tolerance map, it is likely that more than just a tool for carrying out tissue assessment after standing/walking using a current prosthesis will be required. It may also be necessary to develop a way to evaluate skin quality at one point on the residual limb relative to all other points on the residual limb, with other variables kept constant. In other words, temperature related change that is measured in tissue after walking/standing using the patient's current prosthesis would reflect both residual limb skin quality and interface stresses induced by the current prosthesis. It will likely be necessary to develop a way to eliminate the interface stress variable (i.e., make the stress uniform) so that thermal recovery time reflects only skin quality. To address this concern, it would be helpful to create a controlled uniform stress application device.

Such a system exists in the prosthetics industry, although it is not used for assessing skin quality of a residual limb. The Icecast™ (Iceross, Reykjavik, Iceland) is used to make total surface bearing prostheses. A low uniform pressure is applied via an elastomeric liner covering the limb, and this information is used to create an appropriate socket shape. In its most recent version, the device applies pressures to regions of the residual limb. It appears that a higher pressure than this system can provide (5.4 to 10.7 kPa) would be needed for meeting the needs of the desired system. Accordingly, it would be desirable to build and evaluate a system that can provide the necessary pressure levels.

### SUMMARY

This application specifically incorporates by reference the disclosure and drawings of the patent application identified above as a related application.

Infrared (IR) imaging can be used to produce a “map” of thermal recovery time (TRT) for a residual limb. After an amputee has been standing or walking in place for 5 minutes using a prosthesis, TRT is the time interval for a local residual limb temperature to achieve 70% of its maximum value during a 10-minute recovery period. This tool provides a much more objective indicator of potential tissue breakdown in the residual limb than currently used techniques. The success of TRT assessment in this manner is likely to be consistent with a related measurement success in the bedsore area, as noted above.

TRT measures characteristic features of tissue that are related to blood flow. It is important to emphasize that TRT should be used to assess the difference in temperature over time, not absolute temperature. Variables that can affect temperature change include tissue metabolism, and thermal energy changes within the vasculature. Analytical models have shown convincingly that although tissue metabolism can cause significant temperature changes, the time course is very slow and is far less than the sampling intervals that would be used to assess TRT in the residual limbs of amputees. Temperature changes associated with blood flow change, however, are much faster, and are consistent with the sampling intervals that are desired. An increase in temperature indicates initiation or increase in blood flow, while a reduced temperature indicates cessation or a decrease in flow. As defined above, TRT thus reflects the change in blood flow over time after an applied stress is released. A tissue that has a short TRT reperfuses quickly, while tissue with a long TRT reperfuses more slowly.

It appears that interface stress magnitude is much more strongly related to TRT than to temperature magnitude. Since activity is the principle source of breakdown in the patient population of interest here, since repetitive stress is considered the crucial feature leading to breakdown, and since few amputee patients have the disorders for which a relationship between temperature magnitude and future ulcerations was demonstrated, TRT is a better choice for assessment of tissue breakdown in amputee patients than temperature magnitude. Sites on a residual limb that are conditioned to repetitive load would be expected to be more adapted and more load tolerant than sites that are not well-conditioned to load. Accordingly, adapted sites have shorter TRTs than non-adapted sites. This concept is important in the interpretation of TRT data because it adds to an understanding of the link between TRT and adaptation and extends the existing understanding of the link between TRT and tissue breakdown. On subjects using clinically-deemed, acceptably-fitting prostheses, the present novel approach can be employed to assess whether residual limb tissues at sites of high socket rectification, i.e., sites that are subjected to continual high stress, have adapted to decrease TRT durations relative to other locations on the residual limb.

An accurate residual limb tissue tolerance map can be achieved by using both TRT response to uniform load and TRT response to standing/walking conditions while a patient is wearing a current prosthesis. These two tests each provide different information. By considering results from both tests, it is possible to accurately predict how a site will respond to increased interface stress, i.e., increased socket modification. A site that has short TRTs under both uniform load and while a patient is standing/walking using a current prosthetic socket is expected to be both very tolerant compared to the rest of the limb and also very tolerant to interface stresses from the current socket. This site should thus adapt to increased load, i.e., greater socket rectification. A site with short TRTs under uniform load and long TRTs while the patient is standing/walking using the current socket should be a relatively tolerant site but would be highly stressed in the current socket and thus, might either undergo an adaptive response, or break down. It would thus not be prudent to increase socket rectification at this site. A site with long TRTs under both uniform load and while the patient is standing/walking using a current socket is relatively intolerant and overstressed and thus is a strong candidate for imminent breakdown unless modification is decreased. Sites with long TRTs under uniform load and short TRTs while the patient is standing/walking using the current socket are relatively intolerant but are not appreciably stressed in the current socket and thus, are untested. Modification can be increased at these sites, but should be done cautiously.

More specifically, an exemplary method is disclosed for assessing a response of tissue to stress. Stress to the tissue is applied to the tissue being evaluated. Immediately after the tissue is no longer stressed, thermal images of the tissue are collected over a time interval. The thermal images are processed to determine temperature change data for the tissue over the time interval, and the temperature change data over the time interval are automatically evaluated to characterize the response of the tissue to the stress that was applied.

The step of collecting the thermal images include the step of collecting both thermal images produced in response to light received directly from the tissue, and thermal images produced in response to light from the tissue that has been reflected from a reflective surface, such an IR reflective mirror. Control thermal images of a control tissue site that has not been stressed can be collected over the time interval during which the thermal images of the tissue are collected. The control thermal images are then processed to determine control temperature data, and the temperature change data for the tissue under stress are compensated for changes in the control temperature data over the time interval, since these changes are not related to the response of the tissue to stress. Instead, changes in the control temperature data are likely caused by ambient conditions (e.g., drafts) that are experienced both at the control tissue site and at the tissue that was stressed.

The method can further include the step of affixing a plurality of markers to the tissue the stress was applied. Also, visual images of the tissue can be captured over the time interval during which the thermal images were collected of the tissue that was stressed. The plurality of markers is then used for aligning the thermal images and the visual images and for providing an indication of tissue response at specific sites on the tissue.

The step of automatically evaluating the temperature change data over the time interval can include the step of dividing corresponding portions of the thermal images into a plurality of regions, wherein each region corresponds to a specific size pixel area. Then, based upon the temperature change data, the TRT can be computed for each region. A visual map of the tissue can then be created and can indicate the TRT for each of the plurality of regions of the tissue. This visual map thus indicates a rate of recovery of the tissue in each region, after the stress is no longer being applied.

The step of automatically evaluating the temperature change data over the time interval further can also include the step identifying one of a plurality of different characteristic blood flow types for the tissue in each region, based on the temperature change data over time for the region, and indicating the characteristic blood flow type for the tissue in each region on the visual map.

The step of automatically evaluating the temperature change data over the time interval further can further include the step of indicating a prospective condition of the tissue, if the tissue is periodically subjected to substantially the same stress that was applied just before the temperature change data were collected.

The tissue can be on a residual limb of an amputee who uses a prosthetic socket that is worn on the residual limb. In this case, the step of applying the stress to the tissue can include the step of causing the amputee to engage in a specific activity for a defined period of time while wearing the prosthetic socket on the residual limb. This approach is used to assess an effect of the stress applied by the prosthetic socket on the tissue of the residual limb during the specific activity. The specific activity can, for example, be standing while wearing the prosthetic socket on the residual limb, or walking-in-place while wearing the prosthetic socket on the residual limb, or walking up and/or down stairs.

In addition, the method can include the step of applying a uniform stress to the tissue of the residual limb for a specific period of time, while the prosthetic socket is not being worn on the residual limb. The same steps that were carried out above are then repeated to evaluate the effect of the uniform stress on the tissue. Any difference in the response of the tissue to the stress that was applied as a result of wearing the prosthetic socket on the residual limb can be compared to the uniform stress that was applied to determine whether the response of the tissue is due to interface stress caused by the prosthetic socket, or due to tissue quality. The tissue of the residual limb can also be categorized in one of a plurality of different categories, based upon the response of the tissue on the residual limb to the uniform stress and to the stress caused by wearing the prosthetic socket on the residual limb. For example, the category can indicate whether a region of the tissue is: adaptable, indicating that the tissue can adapt or is tolerant to the stress applied; highly stressed due to the stress applied by the prosthetic socket that is worn on the residual limb, so that the tissue might adapt or might breakdown; experiencing a low level of stress due to the prosthetic socket that is worn on the residual limb, but comprising relatively weak tissue; or, at risk of imminent breakdown.

The uniform stress can, for example, be applied by exposing the residual limb to controlled pressure (e.g., a pressure that is controlled to be either greater or less than ambient pressure) for the specific period of time, or rubbing the tissue of the residual limb with a mildly abrasive material for the specific period of time (which is considered “uniform” in the sense that it does not apply stress only to points of contact, such as occurs when an amputee is wearing a prosthetic socket that does not fit properly).

The evaluation of the response of the tissue to stress can be used to determine how to create a new prosthetic socket design for the residual limb that fits the residual limb better than a current prosthetic socket. It can also be used to select components (e.g., suspension, prosthetic foot, ankle, knee, volume management system), control components settings (e.g., ankle power in an active ankle, vacuum level in a vacuum assist device), and for many other purposes.

The method can also include the step of selecting either prosthetic components and/or settings for the prosthetic components, as a function of an effect of the stress applied by the prosthetic socket on the tissue of the residual limb, based upon the effect of the stress determined by the present approach.

It must be emphasized, that this new approach can also be applied to other applications where understanding tissue response to stress is relevant, including seating, shoe and insert design, orthotics, and other disciplines.

Another aspect of this new approach is directed to a system for assessing a response of tissue to stress. The system includes a thermal imaging device that produces thermal images in response to infrared light and which is configurable to collect thermal images of tissue that has just been subjected to stress, over a time interval, and a computing device that is coupled to the thermal image device, to receive and store the thermal images. The computing device carries out functions that are generally consistent with the steps of the method discussed above.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A illustrates an exemplary digital photo of a residual limb taken on June 20 (left panel), and TRT data taken at that date (right panel), wherein the dark boxes for the TRT data indicate short TRT intervals, the light boxes indicate long TRT intervals, and the numbers in each box identify a shape of the thermal response curve for that site, as discussed below, and where the two white boxes indicate the site where tissue breakdown is imminent, although no skin irritation is visibly evident (the white dots on the limb in the left panel are markers used to monitor subject movement);

FIG. 1B illustrates a digital photo of the residual limb shown in FIG. 1A and was taken on July 25 of the same year, wherein a visually apparent tissue breakdown site is noted by an arrow and corresponds to the location of the two white boxes in the TRT data taken on June 20;

FIG. 2 (Prior Art) is a graph from a study by another researcher (Meijer) illustrating thermal response results and showing a strong correlation between a risk of skin breakdown (R) and a thermal recovery time, in minutes;

FIGS. 3A and 3B respectively illustrate an exemplary infrared (IR) imaging camera that can be used for collecting TRT data, and a subject seated in a wheelchair with a residual limb posteriorly supported by a wheelchair pad for imaging with the IR camera;

FIG. 4 is a graph showing examples of four different types of curves observed in thermal response data for corresponding expected blood flow in amputee subjects;

FIG. 5A illustrates an exemplary digital photo (left panel) taken on May 3 for a subject and the TRT data (right panel) taken on the same date, wherein dark boxes in the data indicate short TRT intervals, light boxes indicate long TRT intervals, the numbers in the boxes identify the shapes of the thermal response curves associated with the site of that box, and the white boxes indicate a site of imminent tissue breakdown (indicated within the circle on the left panel), although tissue breakdown was not visually evident at that time (the white dots are markers that are visually evident);

FIG. 5B is a digital photo of the same residual limb taken on June 7, wherein a visually apparent tissue breakdown site is indicated in the circle by an arrow (note that another site shown in FIG. 5A further distally on the limb shows an extended TRT interval, but in FIG. 5B, did not visually show breakdown);

FIG. 6A is an exemplary digital photo (left panel) taken on July 8, of a residual limb of a subject, and the corresponding TRT data taken at that time (right panel), wherein the dark boxes in the data indicate short TRT intervals, the light boxes indicate long TRT intervals, the numbers in the boxes identify the shape of the thermal response curves for the site of those boxes, and white boxes appear where the TRT data indicate an imminent tissue breakdown, although none is visually evident (the white marker dots are used to monitor motion of the limb in another part of the study);

FIG. 6B is an exemplary digital photo (taken on August 5) of the residual limb of the subject of FIG. 6A and includes an arrow indicating a visually apparent tissue breakdown site;

FIG. 6C is an exemplary digital photo (taken on September. 2) of the residual limb of the subject shown in FIGS. 6A and 6B and visually indicates that the tissue breakdown site has cleared;

FIG. 7 is a digital photo of an exemplary embodiment of a uniform stress chamber in which a residual limb of a subject can be inserted within an elastomeric sleeve and exposed to an elevated air pressure applied between the sleeve and the chamber wall, while a distal end plate prevents the elevated air pressure from forcing the limb from the chamber;

FIG. 8 is an exemplary digital photo showing TRT data results (anterior view of right limb) from a uniform stress chamber test of an amputee, wherein the shortest TRT duration are indicated by dark boxes at the patellar tendon (within the ellipse) and anterior lateral distally (within the circle);

FIG. 9 illustrates a plurality of digital photographs showing TRT data for an adaptation study of a non-amputee, wherein the area subjected to stress is indicated in the photo within ellipses, and where the results show a period of lengthening TRT (days 1-8), following by shortening TRT (days 9-10);

FIGS. 10A and 10B respectively illustrate a side view of an exemplary system that includes a chamber for applying a uniform stress to a limb, and an isometric view showing the chamber mounted in apparatus used to adjust its position and alignment;

FIG. 11 is a cross-sectional view showing instrumentation for testing stress distribution on a residual limb model that is positioned in another exemplary embodiment of a stress chamber, wherein interface stress transducers that monitor both pressure and bi-directional shear stress are mounted inside the socket, and their sensing surfaces are disposed in the plane of the interface;

FIG. 12 is a digital photo that illustrates a subject positioned next to an IR-reflective mirror so that both an anterior-lateral and an anterior-medial surface of a residual limb can be simultaneously imaged;

FIG. 13 is a block diagram that characterizes local tissue response based on TRT data collected as described herein;

FIG. 14 is a schematic view illustrating how an IR camera is used in connection with one (or, optionally, two) flat optical mirrors to capture two (and optionally, three) images of a residual limb from different directions (it is noted that the third view is not essential, but does provide useful information about the condition of tissue on the third side of the residual limb); and

FIG. 15 is a flowchart illustrating exemplary steps for carrying out part of the novel procedure disclosed herein for determining TRT data for a limb from a collection of image files for the limb.

### DESCRIPTION

### Figures and Disclosed Embodiments are not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

### Instrumentation

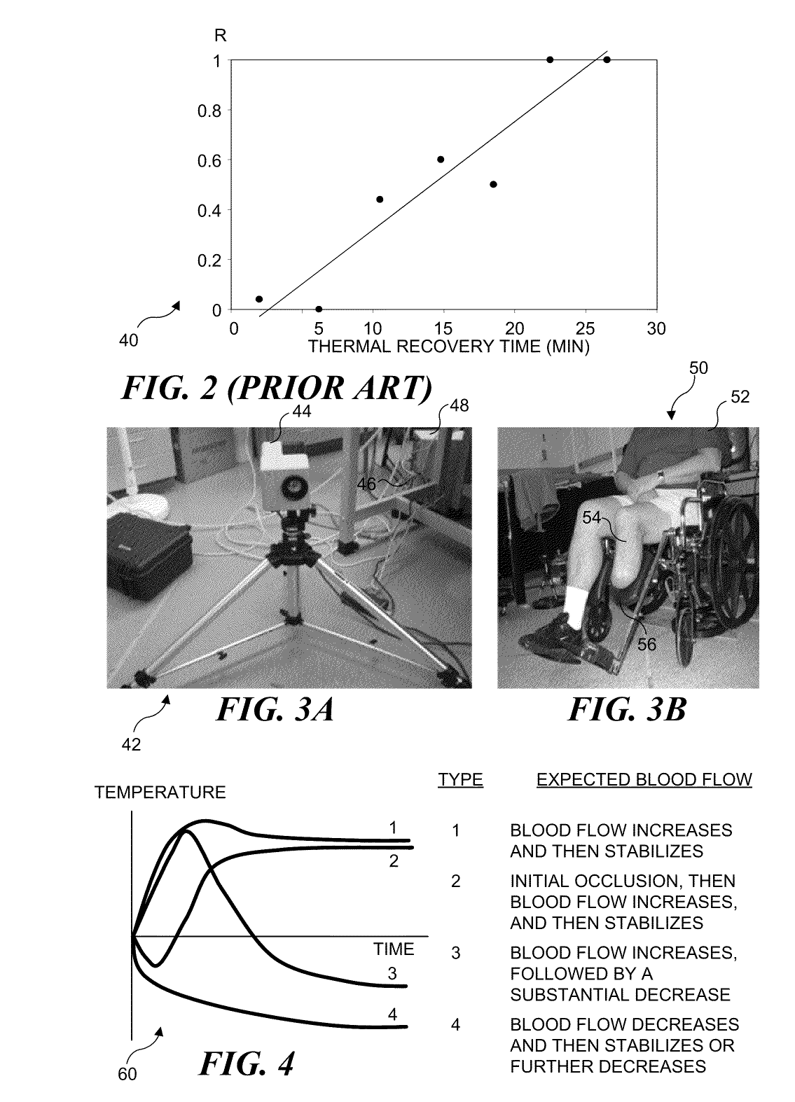

The present novel approach uses an IR imaging system to provide a high-resolution TRT image of an entire region of a limb, thus enabling regions of imminent breakdown to be identified rather than just measuring temperature at single points as was done using a thermocouple during the prior art thermal response study done by Meijer (FIG. 2).

As shown in FIG. 3A, a Thermacam SC3000™ (Flir Systems Inc., North Billerica, Mass.) IR imaging system 42 was employed in an exemplary embodiment of the present system, since it has appropriate thermal and spatial resolutions, sampling rates, and wavelengths for this work. The imaging system includes an IR camera 44, signal processing box 46, and a computer 48 (with a custom image-processing card—not separately shown). In general, an IR camera can be used to assess a material's ability to absorb and radiate energy. Unlike in previous products where IR cameras have required liquid nitrogen continually be provided for cooling, modern IR cameras are cooled using the Stirling cycle. Stirling cycle cooling employs the expansion of gases to produce temperature gradients, making the IR camera easier to use and more temperature sensitive. Data can be collected continuously at up to a 120 Hz sampling rate for an interval up to the storage size available on the hard disk. In this research, each image was approximately 155 kB in size, and sampling was typically conducted at 1 Hz for a 10 minute interval; thus, 93 MB of storage space was typically required per trial. Faster sampling rates and longer sampling intervals are possible if needed in the proposed research, and all of these parameters should be considered merely exemplary and not in any way intended to limit the scope of the present approach.

Although manufacturer specifications were provided, additional tests were conducted to evaluate sources of error relevant to this application of the IR imaging system. Resolution, drift, distance, and incident angle influence were evaluated. Images were taken of a warm object, i.e., a solid aluminum cylinder warmed in a water bath, to determine measurement variability. Over the 15-minute tests, the temperature difference between two points on the object varied by 0.04° C. The standard deviation of the temperature was 0.013° C. To test for camera drift, temperature data were collected at a rate of 1 Hz for 24 h. The temperature of the test object varied by 1.00° C. throughout the test, expected due to air currents in the room, but no drift (i.e., a continuous temperature change in one direction) was evident. The distance between the camera and test object was varied from 1.0 to 1.5 m to test for distance dependence. When the camera was focused at each distance, no temperature difference was measurable. Without refocusing, the average temperature dropped by 0.30° C. for the 0.5 m increase in distance. Thus, it is important to focus the camera properly.

To test for angle dependence, a hollow aluminum cylinder with a bottom of diameter 8.9 cm was filled with warm water (32° C.). A nylon stocking was placed on the cylinder so that the emissivity approximated that of skin. The edge temperatures were compared with the central temperatures to determine performance at angles less than 90°. The test showed the camera was within a 0.04° C. range from 22° to 90°. Temperature differences (0.10° C. to 0.30° C.) were observed for angles from 7° to 22°. A 0.80° C. temperature difference was seen from angles of 4° to 7°, and a 2.40° C. temperature difference was observed at angles of 0° to 4°. Thus, data produced at an angle within a 22° to 90° range were considered reliable.

### Animal Studies

An extensive animal study was conducted during the first 2½ years of this research in an effort to establish a morphological correlate for TRT. During these tests, 64 Landrace/Yorkshire pigs (30-35 kg at the outset of the study) were tested. After one week to acclimate to the housing environment, each pig was subject to the following protocol: The animal was anesthetized using isofluorane by inhalation and the hair was shaved from the test area, i.e., the lateral hind limb. The skin to be stressed was cleaned with an antibacterial agent (Hibiclens™) and sodium chloride irrigation (0.9%, USP), and dried thoroughly. A TRT test was conducted as follows. For a 5-minute interval at a 1 Hz frequency, the region was subjected to cyclic pressure and shear stress profiles comparable to those measured clinically on transtibial amputee subjects. This magnitude was selected because preliminary testing showed the magnitude to be sufficient to induce a reasonable TRT response. A custom closed-loop bi-directional load applicator was used to apply the stress. Immediately after the stress was removed, the region was imaged at a 1 Hz sampling rate for 10 minutes using the IR imaging system shown in FIG. 3A. Care was taken to ensure that an unstressed site at least 5 cm away from the test area was included in the image so that it could be used as a control. Data from the control reflected systemic temperature changes or changes due to fluctuations in the room temperature. The control temperature was subtracted from the test site temperature for each time point.

After IR testing, the same site was subjected to repetitive mechanical stress for a 45-minute period using a bi-directional load applicator. This protocol was run each successive day, for up to five days, at one of five load levels, with the highest load level being approximately twice that measured on lower-limb amputees using prosthetic limbs. In preliminary studies, the highest magnitude was shown to induce a clinical grade I response. On the morning after the last day of the load application, TRT data were taken, and then the animal was euthanized and prepared for histological or immunocytochemical analysis. A number of assays were carried out, including: immunohistochemical labeling for macrophages using mouse anti-human CD163 (DAKO™), Vector ABC kit for secondary, DAB for visualization, and hematoxylin as a counterstain; immunohistochemical labeling for macrophages and endothelial cells simultaneously, thus allowing macrophages associated with vessels to be quantified, using rat anti-house CD31™ (BD Pharmingen); TUNEL™ assay for cell death; caspase 3 assay—alternative assay to assess for cell death; labeling of endothelial cells, thus allowing vessel density to be assessed, using Von Willebrand Factor; assessment of VEGF expression; picro-sirius red staining of collagen; labeling of matrix metalloproteinase 1 (MMP-1); and assessment of nitric oxide synthase (iNOS) so as to evaluate vasodilation.

IR data were processed to identify the time point when temperature reached 70% of its maximum value within the trial. This point was defined as the TRT for the test site. Almost all of the thermal response curves were of Type 1 (see FIG. 4). TRT values ranged from 21 to 400 s.

Qualitatively, the assays for VEGF, caspase-3, the TUNEL assay, MMP-1, and iNOS showed minimal label and thus, could not be evaluated in a quantitative sense. Qualitative evaluation showed no differences for sites with short TRTs vs. long TRTs. Macrophage densities associated with and not associated with vessels also were assessed qualitatively, and no difference was observed for these features, between samples with long TRTs vs. short TRTs.

To determine densities of vessel features, including cross-sectional area, number, and total area, a quantitative morphological analysis method was used. Digital images of sections sampled at regular intervals were superposed with a grid and then labeled or stained circular or elliptical structures indicative of blood vessels within consistently spaced squares in the grid were counted. All data were normalized to the tissue volume used for sampling.

Results using linear models showed extremely weak correlations between TRT and any of these features. There simply was no meaningful relationship. Thus, despite substantial effort, a physiological correlate for TRT could not be identified.

It is possible that the morphological changes were simply too small, i.e., below the noise level in the data. Or, the study duration was too short, and the morphological changes did not happen over this short term. The load levels may have been too low. Or possibly, since the animals were young, they might have quickly adapted to the continual stress, i.e., their response was too quick for the sampling interval used. (Older animals would have been larger and not physically manageable for this protocol and thus, were not used here.) Although this finding might initially appear unfavorable, it is not necessarily so. The most important result from these animal studies is what did not correlate with TRT, and this result helps to provide insight into what TRT might reflect.

Having now conducted and gained tremendous insight from studies on amputee subjects (as described below), there seemed little justification to continue pursuing a morphological correlate using an animal model at this time. The reason is because of what TRT appears to reflect. Based on 4+ years of experience at this type of assessment, it seems likely that TRT reflects something that is not well understood physiologically, happens quickly, is difficult to measure morphologically, but is extremely relevant clinically to prosthetics. It seems likely that TRT reflects pre-injury stages of a tissue adjusting its local blood flow according to its immediate local needs. Thus, a region that is in imminent trouble and needs more nutrients (oxygen, proteins, etc.) from the vasculature to modify or adapt itself to the continual stress conditions it is experiencing, will signal for a temporary increase in blood flow, i.e., for an interval after stress is applied. Because this response is a very initial response, it is apparent only immediately after stress application and is not measurable on a continuous basis. How the vasculature modifies and adapts via vasodilators or other means during this pre-visible-injury stage is a fascinating physiological question. How the tissue uses nutrients and adapts its structure in the longer term to handle the continual stress or slowly breaks down into an injury is likewise a fascinating physiological question. The answers to these questions are not yet known. Accordingly, pursuing a concrete physiological correlate to something that is not known does not appear warranted and is not reasonable; it would be somewhat of a “fishing expedition.” It appeared more worthwhile to pursue a path that may not only lead to a clinically relevant tool to treat people with amputation, but will also likely help to identify a physiological correlate in the future.

TRT is unique. It offers something that cellular and molecular analysis of an animal model cannot—a measurement on living human subjects at risk. TRT has potentially a crucial role in contributing direction to the above physiological questions, gaining insight into blood flow response during pre-visible-injury tissue breakdown and adaptation in amputee prosthesis users. It also potentially provides a useful clinical instrument. The instrument not only can become a useful fitting tool, it also can tremendously enhance understanding of differences in pre-visible injury responses for different subject populations, diseases, and treatment protocols, information that would most certainly help target the physiological aspects of pre-visible injury of most relevance to investigate at a cellular and molecular level.

### Amputee Subject Studies

Subjects: Nine transtibial amputee subjects fully participated in this research (eleven enrolled, but two dropped out after 1 month). They ranged in age from 24 to 74 yr. Two subjects were otherwise healthy, while the other seven had various complications including: high blood pressure, a history of polio, diabetes, persistent pneumonia, peripheral vascular disease, or Larson's syndrome. All subjects wore a prosthesis for at least 7 hr/wk. Informed consent was obtained before any study procedures were initiated.

Protocol: After arriving at the lab, a subject sat for 10 minutes with the prosthesis donned so as to achieve a homeostatic condition. The prosthesis was then removed, and 3 mm diameter markers with sticky backings were applied to the residual limb (9 to 13 markers). The markers, which are made of a material that is visually evident in the images made using the IR camera and also visually evident in conventional digital photos of the residual limb, were necessary during post-processing to correct for subject movement. The markers chosen for use in this study had low emissivity and high reflectivity. It was important that the markers were well distributed throughout the residual limb region of interest.

A series of six trials was conducted over an approximately three-hour period. For each of three views (lateral; medial; and either anterior (eight subjects) or posterior (one subject) depending on which was of greater clinical interest), one standing and one walk-in-place trial were conducted. The ordering of the views was selected randomly. Because analysis showed redundancy in the lateral and anterior as well as medial and anterior views, only lateral and medial views are needed. During a standing trail, the subject stood wearing the current prosthesis with approximately equal weight bearing for a 5-minute duration, holding lightly onto a support, if necessary. The subject then sat in a wheelchair, the prosthesis was quickly removed, and IR imaging was conducted for 10 minutes.

FIG. 3B is an exemplary digital photo 50 that illustrates a subject 52 seated in a wheelchair, with a residual limb 54 supported on a wheelchair pad 56. IR and normal light imaging of the subject's residual limb was thus conducted with the subject in the wheelchair as generally shown in this digital photo.

During a walking-in-place trial, the subject, wearing his/her current prosthesis, stepped forward with the prosthetic limb, then lifted and swung forward the contralateral limb. The subject then shifted weight contalaterally so that the prosthetic limb lifted off the ground. The subject then reversed the motion, stepping backwards to return to the starting position. This process was repeated for a 5-minute interval. A metronome was used to ensure a consistent cadence, using a setting consistent with the subject's typical walking cadence. The subject then sat in a wheelchair, the prosthesis was quickly removed, and IR imaging was conducted for 10 minutes. At the conclusion of IR imaging, a digital photograph was taken of the residual limb at a position immediately adjacent to the IR camera. Before each new trial, the subject rested comfortably with the prosthesis on for 10 minutes. This process was repeated until all six trials were completed.

Walking-in-place as opposed to ambulatory walking trials was conducted because of the effects of full ambulation on the data. Preliminary testing showed that ambulation caused the entire body to experience a TRT response that was much more substantial than the response to a local stress applied to the residual limb, the response of interest. Ambulation essentially washed out the data of interest. By walking-in-place, the subject's heart rate was not substantially increased, less energy was expended, and the whole body response was reduced. The local response was thus apparent in the TRT data.

It appears that the standing and walking-in-place tests reflect different interface stress conditions, much in the same way that standing and ambulating during prosthetic fitting provide different evaluations. Standing with equal-weight-bearing tends to induce lower interface stresses that can occlude blood flow locally, since the loading is static rather than dynamic. Walking in place induces higher interface stresses, but these stresses are dynamically applied, meaning that reperfusion might occur during each cycle. Both were tested in this study because both conditions were of clinical interest.

Both before and after the session, the subject's residual limb was inspected thoroughly by the study prosthetist for signs of breakdown or injury. Breakdown was very conservatively defined as any reddening or other sign or irritation. Affected sites were noted and digital photographs taken of the affected regions.

This protocol was run on each subject once a month for at least a six-month interval. A total of 336 image trials were conducted.

Data Processing The intent in post-processing the data was to create a TRT image of the residual limb. First, subject movement during scanning needed to be corrected in the data. In each image (there were 600 images for a 10-minute trial) the centroids of the markers placed on the limb were identified using image processing code written in Matlab™ (Mathworks, Natick, Mass.). An optimization procedure was then implemented to create six-directional spatial transformations for each limb, thus establishing subject movement in all six directions from one time point to the next. By having the markers well distributed throughout the region of interest, a very accurate correction algorithm was created. Evaluation tests conducted using image sets with randomly generated displacements (for all six directions) showed that the algorithm realigned the residual limb within 1 pixel resolution.

Next, the residual limb region facing the IR camera that was within the acceptable angle of incidence range (22° to 90°) and of clinical interest for TRT characterization was identified and broken up into regions of 5×5 pixels. A pixel at the border between regions was not included in either region so as to minimize overlap between regions. The marker dots were identified, and those pixels were not included in the analysis. The 5×5 pixel region size was selected, not only because it proved appropriate for clinical analysis, but also because it allowed a marker to be removed, while still leaving sufficient pixels in the region for analysis; however, it should be understood that this detail is not to be considered a requirement or limitation on the scope of the present approach, since different size pixel regions can be employed and none of the pixels must necessarily be dropped. Typically, a 5×5 pixel region was of dimension approximately 6×6 mm. The average temperature value within the region was calculated for each time point. The maximum and minimum temperature within the 5×5 pixel region were also calculated for each time point, since they might also prove useful in TRT analysis. At each time point, all temperatures were subtracted from the temperature at a control region disposed over the patella. This site, which was under the prosthetic liner but was not stressed by the socket during standing or walking-in-place reflected temperature changes due to changes in room temperature and to the subject's skin cooling from removing the liner. Thus, by subtracting the temperature of the control site from each site that was under stress, influence of these two effects was eliminated from the data of interest. A temperature vs. time curve was created for each region. The shapes of the curves were then classified into one of four types, as indicated by a graph 60 in FIG. 4, as follows:
Type 1: temperature rises to a maximum or plateau value;
Type 2: temperature initially decreases, then rises to a maximum or plateau value;
Type 3: temperature rises to a maximum and then decreases below the initial value; and
Type 4: temperature decreases to a minimum or plateau or continues to decrease.

These four types were defined based on experience previously gained in testing amputee subjects. It was expected that in terms of blood flow, the four regions reflect the associated conditions listed in FIG. 4. For Types 1 and 2, the interpretation is consistent with Meijer's, Type 1 representing a blood flow increase and then stabilization, and Type 2 representing initially occlusion, and then a blood flow increase followed by stabilization. These are the two most common types of responses that were observed. Type 3 is similar to Type 1, except that the temperature decreases to a value lower than the initial temperature after socket removal, reflecting a large decrease in blood flow. It is unclear if this large decrease reflects an overcompensation response to the stress. However, this type of curve becomes relevant in discussion of the adaptation results that are described below. Type 4 shows a decrease in blood flow compared to that initially observed, and could represent tissue with minimal blood flow, possibly reflecting vessel damage. However, it could also reflect tissue that was not stressed or minimally stressed, as well. Part of the impetus for developing a uniform pressure chamber is a need to distinguish between these two possibilities. The addition of TRT data after uniform pressure application should make it possible to distinguish between damaged tissue vs. a low stress application.

TRT was calculated for each 5×5 pixel region for curve Types 1, 2, and 3 as the time interval required for the temperature to reach 70% of its maximum value. The reason 70% was used was that in cases where the temperature gradually reached a peak, the time interval in which the maximal temperature was reached was highly sensitive to noise in the temperature data (i.e., the temperature/time slope was low). By using 70% of the maximum, this problem was essentially eliminated. Preliminary testing using values of 60%, 70%, 80%, and 90% produced comparable relative TRT differences among sites; however, a value of 70% was chosen for use in this project, since it produced the most consistent result. For Type 4 curves, the time for the temperature to reach 70% of the minimum temperature value was calculated. However, it is not intended that the choice of 70% is a requirement, since it is contemplated that other percentage values may instead be used, such as 60%, 80%, 90%, etc.

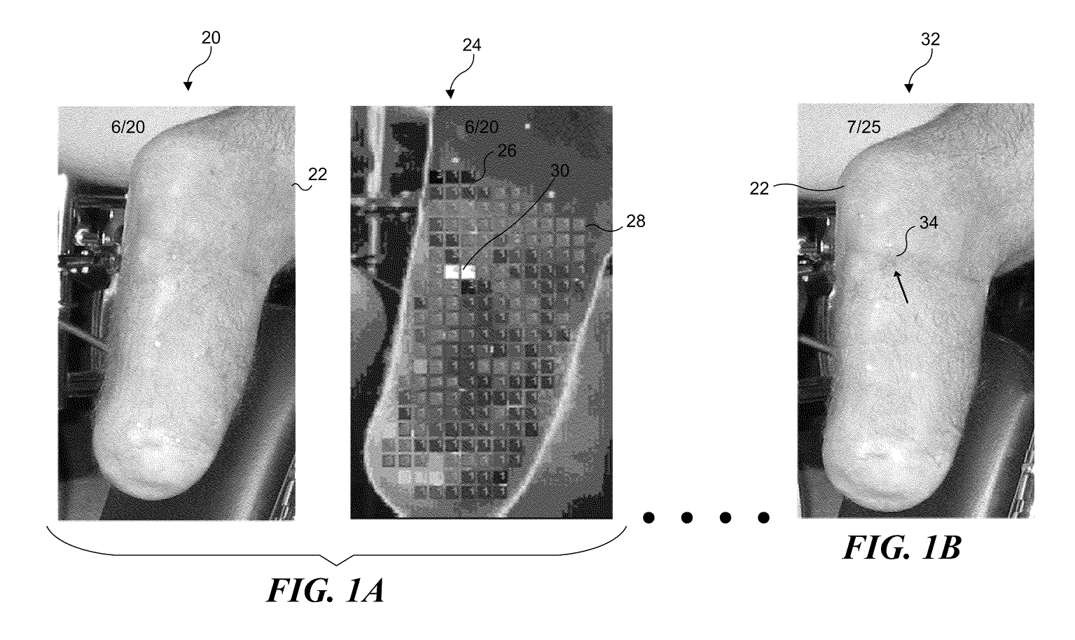

Images were processed and presented so that they showed both the type of the curve and the TRT value for each region, as shown in FIGS. 1A and 1B; 5A and 5B; and 6A, 6B, and 6C. In FIG. 1A, a digital photo 20 shows a residual limb 22, and corresponding TRT data 24. Dark boxes 26 in the TRT data indicate short TRT intervals, lighter boxes 28 indicate long TRT intervals, and the numbers in each box identify a shape of the thermal response curve for that site, as discussed in connection with FIG. 4. Two white boxes 30 indicate the site where tissue breakdown is imminent, although no skin irritation is visibly evident. In digital photo 20 (and other similar digital photos), the white dots on the limb in the left panel are markers used to monitor subject movement are fabricated of a material that is particularly visually evident in the images used for TRT data and also visually evident in the conventional digital photos of the limb. Both digital photo 20 and the images of TRT data 24 were taken on June 20. FIG. 1B illustrates a digital photo 32 of residual limb 22 and was taken on July 25 of the same year. A visually apparent tissue breakdown site 34 is noted by an arrow and corresponds to the location of two white boxes 30 in the TRT data taken on June 20.

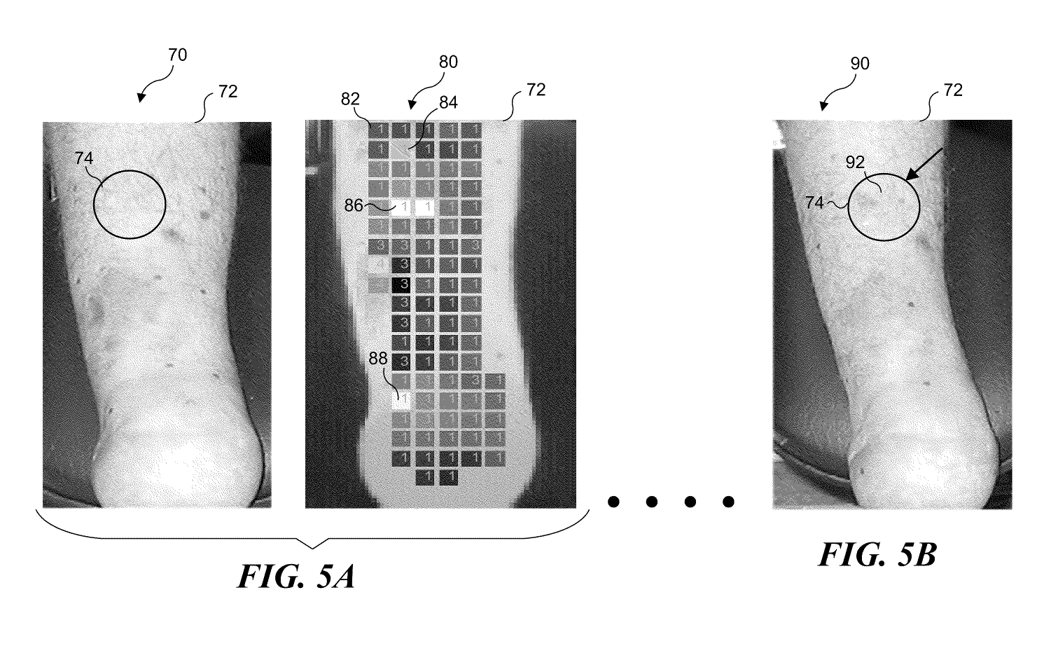

FIG. 5A illustrates an exemplary digital photo 70 (left panel) that was taken on May 3 of a residual limb 72 of a subject and corresponding TRT data 80 (right panel) taken on the same date. Dark boxes 82 in the data indicate short TRT intervals, lighter boxes 84 indicate long TRT intervals, the numbers in the boxes identify the shapes of the thermal response curves associated with the site of that box. White boxes 86 indicate a site of imminent tissue breakdown (i.e., the site within a circle 74 on the left panel), although tissue breakdown was not visually evident at the time digital photo 70 was taken. FIG. 5B illustrates a digital photo 90 of residual limb 72 that was taken on June 7 (same year as digital photo 70). A visually apparent tissue breakdown site 92 is indicated in circle 74 by an arrow. Note that another site 88 shown as another white box in FIG. 5A, further distally on the limb, appears in digital TRT data 80 as an extended TRT interval, but in digital photo 90 of FIG. 5B, does not visually show breakdown.

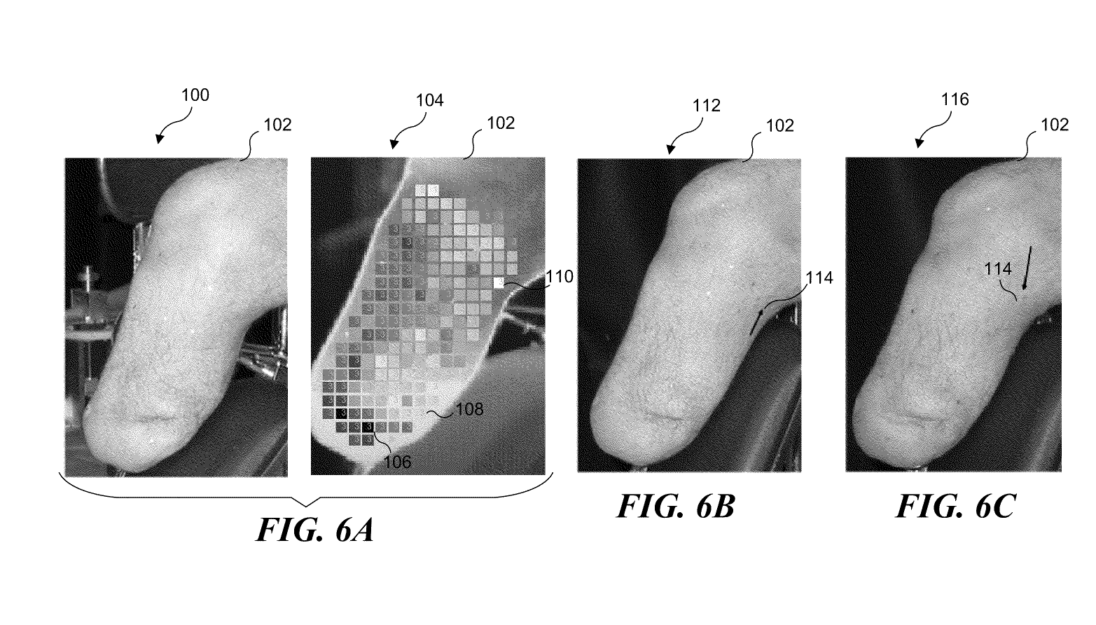

Similarly, FIG. 6A illustrates an exemplary digital photo 100 (left panel) taken on July 8, of a residual limb 102 of a subject, and corresponding TRT data 104 taken at that time (right panel). Dark boxes 106 in the data indicate short TRT intervals, lighter boxes 108 indicate long TRT intervals, the numbers in the boxes identify the shape of the thermal response curves for the site of those boxes, and white boxes 110 appear where the TRT data indicate an imminent tissue breakdown, although none is visually evident in digital photo 100. FIG. 6B illustrates an exemplary digital photo 112 (taken on August 5) of residual limb 102 and includes an arrow indicating a visually apparent tissue breakdown site 114. FIG. 6C illustrates an exemplary digital photo 116 (taken on September. 2) of residual limb 102 of the subject shown in FIGS. 6A and 6B and visually indicates that tissue breakdown site 114 (indicated by the arrow) has cleared.

Thus, when inspecting a processed TRT image for sites of possible imminent breakdown, we first look for light colored regions in the image. Light colored regions indicate long TRTs. If the region and those surrounding it are curve Types 1-3, then this is a site suspect of imminent injury. Type 4 curves are more difficult to interpret because it is unclear if the cooling reflects a poor vascular response or if the region simply received very little stress while the subject stood or walked in place using the current prosthesis. If a Type 4 curve appears in one or two grid regions surrounded by non-Type 4 curves, then this site is likely at risk of breakdown.

TABLE 1
Results from amputee subject study.

| | | Months picked up w/TRT | Standing or |
|---|---|---|---|
| Subject | Location | before visually apparent | Walking-in-Place |
| A | anterior mid-limb midline | 1 | W |
| | anterior distal tibia | 1 | W |
| | anterior lateral mid-limb | 1 | W |
| | lateral distal end | 1 | W |
| B | anterior mid-limb | 2 | W |
| | proximal mid-limb | 1 | W |
| | lateral proximal | came into study with injury | W |
| | lateral mid-limb | came into study with injury | S |
| | lateral mid-limb distal | 0-1 (1 months data out of view) | W |
| | lateral distal | 1 | S |
| | medial proximal | 2 | W |
| | lateral mid-limb | came into study with injury | S |
| | inflamed hair follicle | 1 | S |
| C | distal edge of patella | came into study with injury | |
| | just below distal edge of patella | came into study with injury | |
| | anterior distal end | came into study with injury | |
| | medial proximal edge | 1 | W |
| | lateral proximal | 0 | S/W |
| D | anterior distally near tattoo | 1 | W |
| | anterior proximal lateral | 1 | W |
| | distal end | | |
| | proximal, medial side of midline | 1 | W |
| | anterior distal end | 1 | S |
| | medial pimple | 1 | S |
| | proximal medial sore | 1 | W |
| | anterior lateral distal | 1 | S |
| E | anterior proximal, lateral side | 2 | W |
| | posterior lateral | came into study with injury | |
| | posterior medial | 1 | W |
| F | anterior distal end | 1 | S |
| | mid-limb | 1 | S |
| G | anterior lateral proximal | 1 | S/W |
| | anterior medial proximal | 1 | S |
| | anterior distal lateral end | 2 | W |
| | proximal lateral | 1 | S/W |
| | medial proximal | came into study with injury | |
| H | anterior distal tibia | came into study with injury | |
| | anterior distal tibia | came into study with injury | |
| | mid-limb | came into study with injury | |
| | anterior mid-limb | 1 | S |
| I | anterior distal | 1 | W |
| | lateral distal | 1 | S |

Sites of injury, when identified, and by which loading test.

Results: The nine-person amputee subject study produced interesting results. TRT values typically ranged from approximately 50 to 300 s. There were 30 cases of skin breakdown during the study that were not present at the outset, with breakdown defined by the study prosthetist as clinical evidence of skin reddening or other signs of tissue irritation.

TRT well predicted cases on imminent breakdown, typically 1 month and sometimes 2 months in advance (see Table 1, above). A typical case is shown in FIGS. 5A and 5B where an extended Type 1 curve is measured 35 days before an injury was clinically visible. In FIGS. 6A, 6B, and 6C, an extended Type 3 curve was measured 28 days before injury was clinically visible. Injury was visually clearer at 56 days.

As shown in Table 1, sometimes data from both loading conditions (standing and walk-in-place) identified an imminent injury. In other cases, only one condition predicted the result. This finding presumably indicates that in some cases, only the one condition had sufficiently high interface stresses or stress durations to cause a lengthened TRT response. That condition, done repeatedly by the subject outside of the lab, may have caused the injury. It is revealing that in some cases, static loading identified the injury, while walking-in-place did not. If this interpretation is correct, then the result points to the relevance of static loading, not just dynamic, towards tissue injury.

Interestingly, once breakdown was clinically visible or if breakdown was clinically apparent at the outset of the study, the system did a poor job of showing it. Those sites simply did not “light up” in the TRT image. Since the system is intended to predict imminent breakdown, not show that it is there when it is clinically visually apparent, this result is acceptable. However, this result might help towards an understanding of blood flow bio-response during tissue breakdown. Apparently, increased durations of local blood flow after stress did not continue once the injury was visually apparent.

Imaging was conducted from three views on all subjects for completeness. This practice added considerable time for data collection, compared to imaging from only one direction. To overcome this problem, data can be simultaneously obtained from more than one direction. Data collection time should thus be reduced, and this design enhancement is included in the present exemplary approach.

There were a number of cases of false positives (a total of 32) in this study, i.e., regions where the TRT image predicted imminent skin injury but breakdown did not materialize by 1 to 2 months later. An example is shown in FIGS. 5A and 5B. Possibly the stress was relieved before injury occurred. Or, these sites possibly underwent an adaptive response rather than breakdown. It might be that the initial paths towards breakdown and adaptation are comparable. Eventually a “Y” is reached where the tissue goes one way or the other—it either adapts or it breaks down. It may be that the TRT data are picking up the response before this “Y” has been reached. At this point, this interpretation is highly speculative. However, it is consistent with findings from the bone biomechanics literature concerning trabecular bone remodeling. Micro-fractures occur not only during the initial stages of injury, but also during the initial stages of adaptation. If the tissue adapts, it is subsequently replaced by stronger and more biomechanically appropriate trabecular structures. By understanding how adaptation affects TRT data and combining that with the knowledge of pre-breakdown TRT data, it should be possible to distinguish adaptation from imminent injury.

### Uniform Stress Chamber Studies—Amputee Subjects

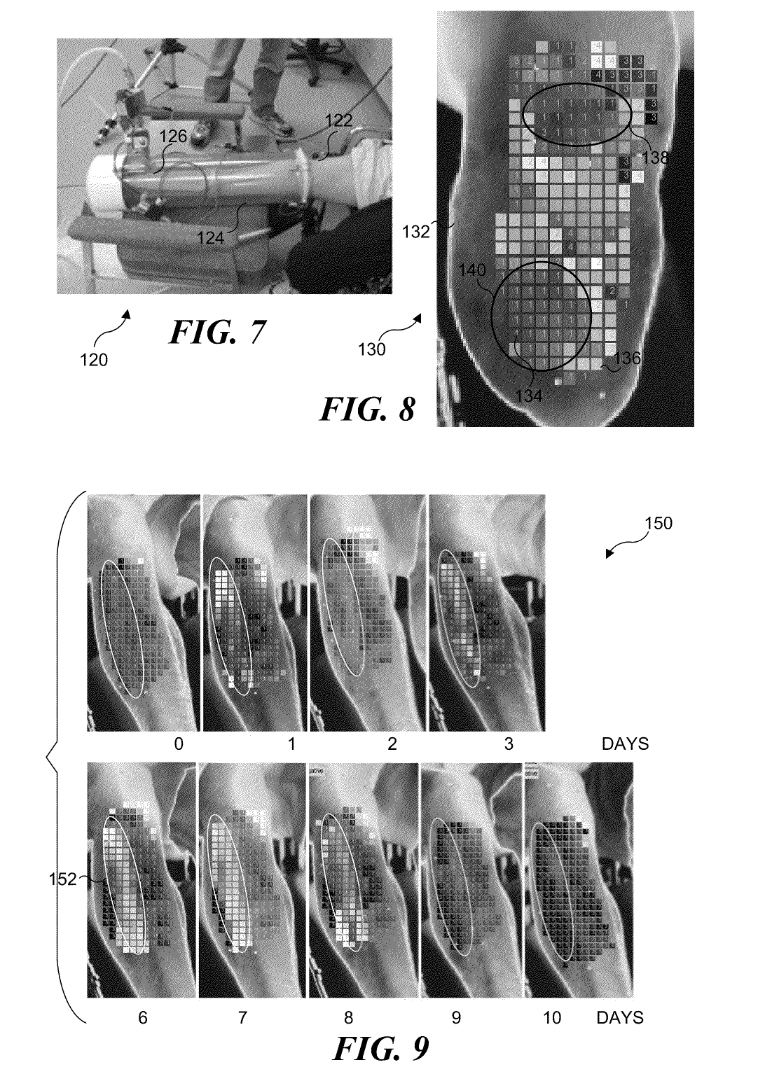

As shown in a digital photo 120 in FIG. 7, a preliminary prototype exemplary uniform stress chamber 124 was created to apply uniform stress to a residual limb 122. The chamber was a plastic (PVC) cylinder with a latex sleeve 126 disposed within it, somewhat similar to the Icecast™ fabrication system.

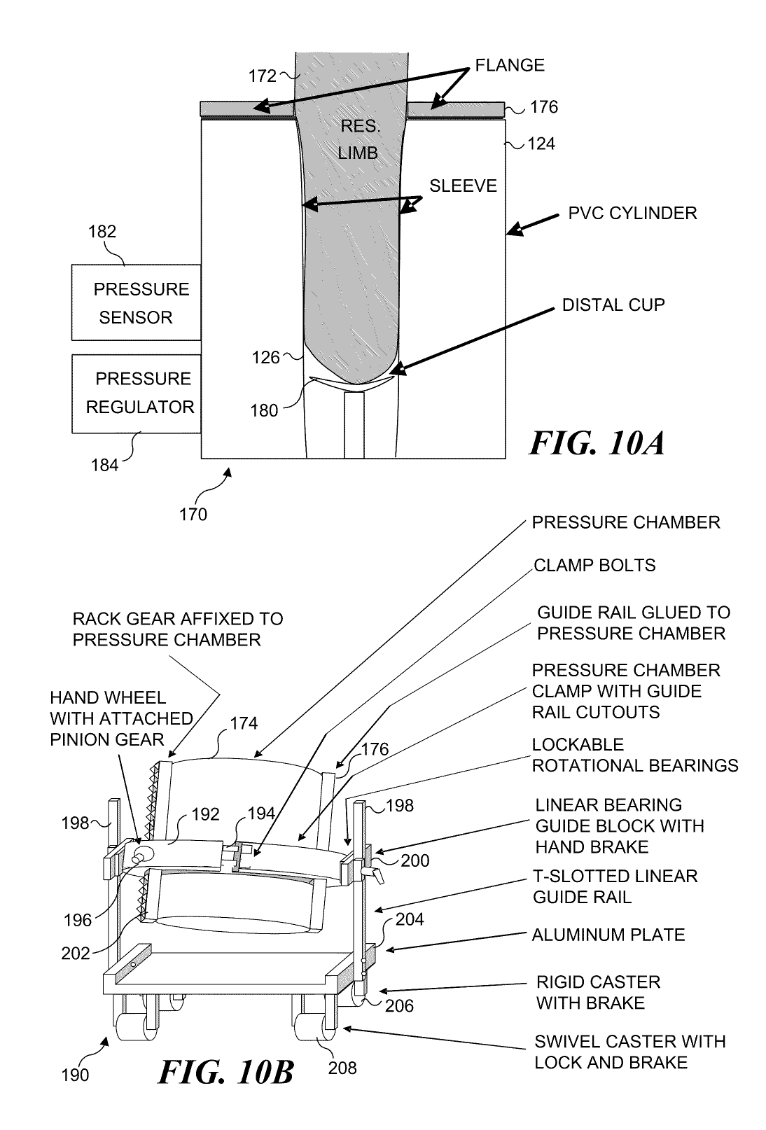

In a schematic illustration 170 shown in FIG. 10A, further details of the stress chamber are illustrated. A distal cup 180 was positioned in chamber 124 so that it could be moved up near to the distal end of a residual limb 172. The plate was necessary so that sleeve 126 did not cover the distal end of the residual limb. If the sleeve did contact the end of the residual limb, the sleeve would push the limb out of the chamber when pressure was applied between the interior of the chamber and the sleeve. Further, use of the plate better simulated the elastomeric sleeve achieving a no-distal-end-bearing condition, which is consistent with most prosthesis designs. A pressure regulator 184 and a pressure sensor 182 were positioned on the cylinder wall and coupled to a personal computer (PC) (Dell Inspiron™, Round Rock, Tex.) that included a data acquisition card (National Instruments, Austin, Tex.), neither shown in this Figure. A closed-loop control system was created and implemented in the LabView™ software program to adjust the applied pressure. The chamber pressure was set to a maximum value between 13.8 and 38.0 kPa, and pressure was applied either statically or dynamically. The chamber was supported by a mechanical frame for stabilization. It should be noted that stress can be uniformly applied with the chamber by subjecting the limb to a pressure in the chamber that is greater than or less than ambient pressure.

Although this initial stress inducing system fulfilled the need for this preliminary study, modifications are contemplated that should make it more effective. The cylinder needs to be shorter but wider so as to reduce the amount of air within the cylinder and thus improve dynamic response. A larger width cylinder 174, as shown in FIG. 10B, should also accommodate subjects with larger-diameter residual limbs. Cylinder 174 is mounted on an alignment frame 190, such that it is adjustable in space and is capable of being lined up with a proximal thigh of a subject. Cylinder 174 has a guide rail 176 that is adhesively attached longitudinally along the outer surface of the cylinder. A pressure chamber clamp (with guide rail cutouts) 192 hold the cylinder in place as clamping force is applied by clamp bolts 194. Before the clamps are tightened, the position of the cylinder can be adjusted by turning a hand wheel 196, which is attached to a pinion gear (not shown) that engages a rack gear 202. The rack gear is attached to the outer surface of the cylinder and extends longitudinally, much like guide rail 176. The cylinder is supported in alignment frame 190 by vertical T-slotted linear guide rails 198. A linear bearing guide block with hand brake 200 can be adjusted and set to locate the cylinder vertically in a desired position. The pressure chamber clamp also can be rotated in lockable rotating bearings (not visible in this Figure) disposed adjacent to the vertical T-slotted linear guide rails. The vertical T-slotted guide rails are attached to an aluminum plate base 204 and the base can be rolled over a supporting surface on rigid casters 206 and swivel casters 208 that are fitted with a locking brake.

Such a design for the stress inducing cylinder and alignment frame will reduce loading on the posterior thigh of a subject during use. The hoses and cables can be positioned so that they are unobtrusive during testing. It is important that the pressure chamber be capable of being removed quickly so that TRT assessment starts immediately after load application.

Results from preliminary investigations using this system on two amputee subjects (six imaging sessions) showed results consistent with expectations (results were similar for both subjects). FIG. 8 is an exemplary digital photo 130 of the TRT data for a limb 132 of one of the subjects. Regions of high load bearing, i.e., the patellar tendon (indicated within an ellipse 138), and the anterior lateral distal end (indicated within a circle 140), experienced relatively short TRTs (identified by dark-colored grid squares 134) compared to other regions on the residual limb with lighter grid squares 136. Because this subject was also a subject in a different study involving interface stress measurement, there is clear evidence that he experienced high stresses in these two regions compared with other locations on the residual limb.

These results are very encouraging in that they are consistent with the expectation that areas of frequent high load bearing show shorter TRT intervals than other regions. It is likely that these areas are more adapted to mechanical stress. The results also suggest that the chamber does indeed apply uniform load. Although results are encouraging, a study characterizing adapted tissue on a larger amputee subject population should be able to confirm this result, and shape differences between the limb and socket can then be compared to the TRT data to confirm the correlation.

### Evaluation of Stress Uniformity

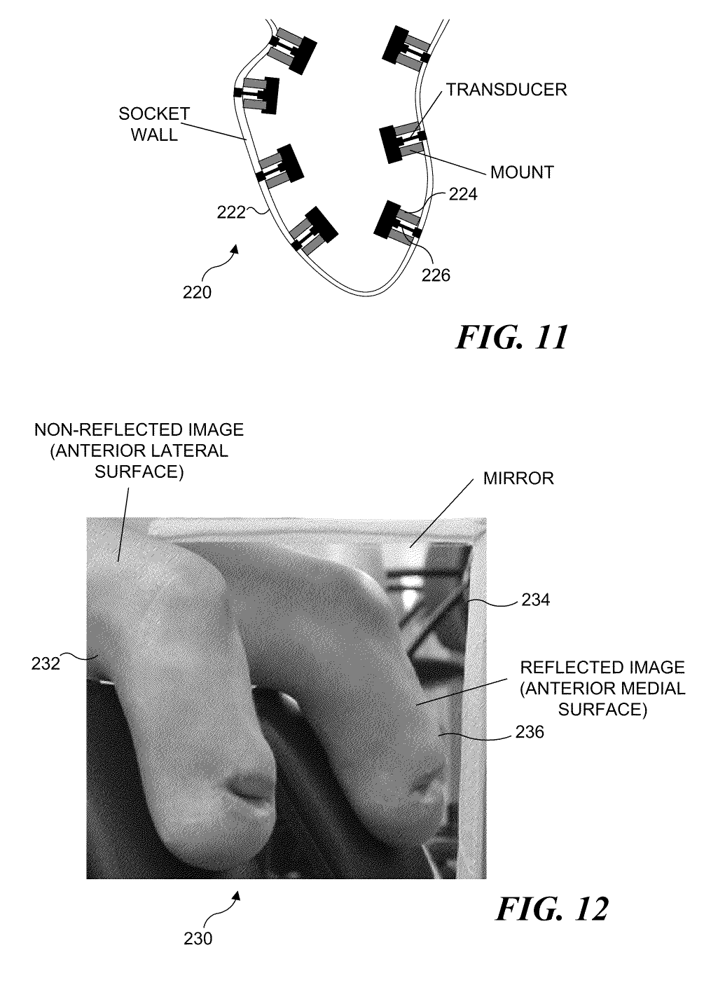

As illustrated by an example of a physical model 220 in FIG. 11, to evaluate the uniformity of stress application, a plurality of these thin-walled, residual limb physical models can be fabricated, instrumented with force transducers 226, and then tested in the stress inducing chamber. The model external surfaces 222 are in shapes of residual limbs of amputee prosthesis users and can be made of Lexan™, a material commonly used for prosthetic check sockets. The intent of having a plurality of different shapes for testing is to mimic the different features of limb shapes that are likely to be encountered during clinical studies. The models can be manufactured using computer-aided manufacturing methods by a local central fabrication facility that produces reliably-shaped check sockets. The residual limb shapes selected are from a computer-aided design and manufacturing clinical database and exemplify a range of shape features often seen in transtibial amputees. For example, Model #1 is conically-shaped, Model #2 is more right-circular-cylinder-shaped with few bony prominences, and Model #3 has many bony prominences.

The models will be instrumented with 15 three-directional force transducers 226 that have been used previously in prosthetic interface mechanics research. The transducers measure both pressure and bi-directional shear stress and are accurate to within 0.5% full-scale-output (FSO). They will be positioned on mounts 224, so that their sensing surfaces are in the plane of the interface, in this case facing the model external surface. The transducers are equipped with custom amplifiers and signal conditioners in the rear of the transducer bodies (not shown). Also not shown are cables that extend from the transducers to a signal conditioning/multiplexing unit such that the transducers (45 channels) are multiplexed onto three signal lines to a data acquisition system, e.g., a PC (Dell Dimension™, Round Rock, Tex.) with a data acquisition card (National Instruments, Austin, Tex.).

To conduct a test, the uniform stress chamber will be put on the model and adjusted as would be done during clinical studies on amputee subject residual limbs. Interface stresses will be monitored at a 175 Hz sampling rate (the current capability of the system) while chamber pressures are applied statically and then dynamically. During static testing, ten pressures in increasing increments between 0 and 48.3 kPa will be applied. 48.3 kPa is the maximum pressure expected tolerable in the chamber. Pressures will be held for at least 1 minute before data are acquired for a 10-second interval. The intent of the delay is to ensure any material relaxation has occurred, and interface stresses are at a constant value. During dynamic testing, sinusoidal pressures (a range of 0.05 Hz to 1 Hz will be tested) will be applied with amplitudes up to 48.3 kPa. During each cycle the minimum pressure will be 0 kPa. After 1 minute to achieve a consistent response, interface stresses will be measured while dynamic pressures are applied for a 1-minute interval.

Interface stress data will be processed using the same procedures as that for interface stress data collected on amputee subjects. Stress vs. time curves will be created for each channel. For static data the mean stress for each pressure level for the 10-second sampling interval will be calculated and comparisons made among the 15 transducer sites. For dynamic pressure application, a mean curve will be calculated for the 60 cycles monitored for each pressure level. Interface stress amplitudes will be compared among the 15 transducer sites. Phase lag will also be investigated but if consistent among all sites, as expected, will not be pursued further.

It is expected that pressures and shear stresses will be relatively uniform given the low stiffness nature of the elastomeric sleeve and the geometric configuration of the system. However, if proximal to distal stress gradients are found or if there is a dependence on underlying model stiffness, then means to overcome them will be pursued. Possibilities include using a different elastomeric sleeve for the chamber, or custom fabrication of the shape of the elastomeric sleeve for each subject using an elastomeric polymer that can be poured (TAP Mold Builder™, TAP Plastics, Seattle, Wash.). Based on interface stress studies that show step-to-step interface stress variations of approximately 5%, differences of 5% or less between sites can be considered acceptable and differences outside that range unacceptable.

### Adaptation Study—Non-Amputee Subject

A preliminary investigation was conducted on a non-amputee subject to assess TRT changes during tissue adaptation to repetitive stress. To condition the limb of the subject, a repetitive pressure and shear stress were applied to the lateral lower leg once a day for 5 minutes. The stress was applied using a towel rubbed briskly over the anterior-lateral proximal lower leg. Once each morning (before a repetitive stress application session was conducted), TRT was assessed on this region.

To conduct a TRT test, stress was applied by having the subject kneel onto a chair such that all the body weight was put onto the lateral, proximal, lower right leg. The subject then returned to a standing position. This maneuver was repeated at approximately a 1 Hz frequency for a 5-minute interval. Immediately after loading, TRT was assessed for a 10-minute interval while the subject sat comfortably.

FIG. 9 includes a series of digital photos 150 showing TRT data results collected over the 1.5-week study interval. The data showed an initial period of lengthening TRT in areas of stress within ellipses 152 lasting approximately eight days. (No data were collected on weekend days 4 and 5, although repetitive stress with the towel was applied on those days.) This lengthening was followed by a two-day period of TRT shortening and a transition from Type 1 curves to more Type 3 curves. These results are very encouraging in that a systematic adaptive change was seen. An initial lengthening of TRT and then a reduction to a value shorter than the initial TRT is consistent with an adaptive response. The change in curve type from Type 1 to Type 3 (see the description of these curves in FIG. 4) might reflect a compensation mechanism that facilitates tolerance to the continual stress.

Although these results are very encouraging, it is expected that further studies may be conducted on the subject population of interest—lower-limb amputees. During such studies, a better controlled means to apply stress so as to ensure consistency may be used.

### IR Camera Setup for Capturing Multiple Images of a Residual Limb

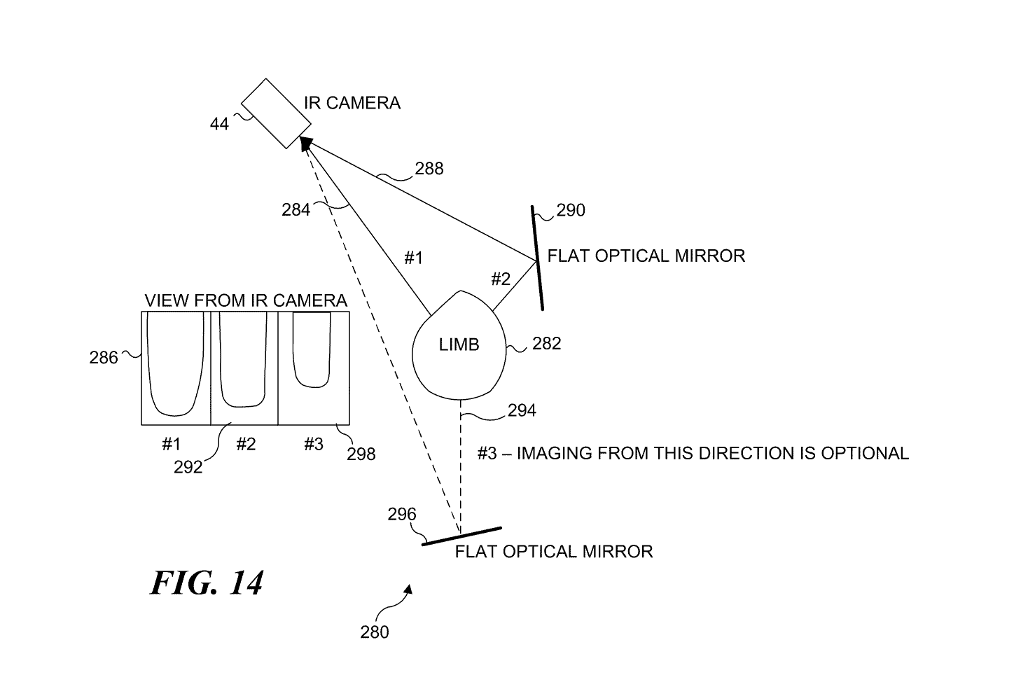

FIG. 14 illustrates a setup 280 for IR camera 44 that uses one flat optical mirror 290 (and optionally, a second flat optical mirror 296) to enable two images 286 and 292 (and optionally, a third image 298) of a residual limb 282 to be simultaneously captured from different directions. The setup enables the anterior aspect of the limb (path #1, where light travels from the limb along a path 284, and path #2, where light travels from the limb, is reflected from flat optical mirror 290 and continues along a path 288) or the entire limb (by optionally adding second flat optical mirror 296, so that light from the limb travels from the limb along a path 294 and is reflected from the second flat optical mirror toward the IR camera, as indicated for path #3) to be imaged at the same time. Optionally, additional mirrors may be added to shorten path lengths or to acquire images from additional orientations. For example, two optically flat mirrors might be used to view the posterior aspect of large limbs since the image may otherwise be too small. The anterior aspect is most relevant because soft tissue problems almost always occur on the front half of the residual limb. However, posterior assessment might be useful in some cases. The image taken along path #3 makes imaging more challenging, because the limb cannot be supported posteriorly during assessment. The camera field of view is broken up into three sections (two sections if only paths #1 and #2 are used), as shown. The camera and mirrors are positioned such that the incident angles of the optical paths are approximately perpendicular to the skin. The images for paths #2 and #3 are smaller because of the longer optical path lengths. Images for paths #2 and #3 are also inverted because they are mirror reflections.

Regular flat optical mirrors are used in the setup to ensure consistent resolution and to minimize distortion. Further, protected-gold coated mirrors are beneficially used because they have maximum reflectivity in the IR wavelength range of interest (8-9 μm) and reflect over 98% of the incident light.

FIG. 12 is an exemplary digital photo 230 that illustrates how a non-reflected image 232 showing the anterior lateral surface of the residual limb of a subject can be captured simultaneously with a reflected image 236 showing the anterior medial surface of the limb, using a mirror 234. To capture the optional third view using an optional second mirror, the subject cannot be seated in a wheelchair as shown in this digital photo.

Protocol for Further Studies: The following protocol is recommended for application of the present approach in further studies. At the time of entry to the study, a study prosthetist will evaluate a subject in the same manner as he/she would a new patient. This evaluation should include collection of the following data for each subject by oral administration of a questionnaire: age, sex, race, date of amputation, cause of amputation, date of delivery of the prosthesis, name of current prosthetist, average weekly hours of prosthesis use, activity level, comfort on a scale of 1 to 10, number of breakdown events in the past one month and in the past one year, and existence of a systemic vascular disease (diabetes, cardiovascular disease, peripheral vascular disease). The study prosthetist should evaluate the residuum for signs of current breakdown, and a photographic record of the residuum should be made.

Subjects should attend two parts for data collection. In the first part, the subject's normal walking cadence (steps/minute) will be assessed, and an IR imaging session will be carried out.

Subjects will be instructed not to drink caffeine or alcohol before coming to the lab for testing on the data collection day. The basis for this constraint is that these two substances may affect vasoactivity and thus TRT response. Upon arrival to the lab, the subject should sit for 10 minutes with his/her prosthesis donned to achieve homeostasis. During this time, subjects will be asked if there have been any changes to their daily routine or health since they enrolled in the study. Then the markers to be used for identification of limb orientation during image-processing will be put on the residual limb, taking care to ensure they are well-distributed throughout the regions of interest. These markers can be selected to be particularly visually evident in the thermal images made using the IR camera, as well as visually evident in the conventional digital photos of the tissue on the residual limb. The subject will then don his/her prosthesis, have his/her weight taken, and then sit for 5-10 minutes with the prosthesis on.

TRT data will be collected after both standing and walk-in-place conditions using the current prosthesis. A subject will stand or walk-in-place for a 5 minutes interval, then immediately sit in a wheelchair and remove his/her prosthesis. IR imaging will be conducted for a 10-minute period. Data will be processed to determine if an imminent breakdown site exists. If not, the second part of the data collection session will be conducted. Alternatively, the second part can be scheduled to take place within approximately one week of the first part.

During the second part of the data collection session, IR imaging will be carried out, but using the uniform stress chamber rather than stand/walk-in-place testing using the current prosthesis. The subject will sit for 10 minutes with his/her prosthesis donned to achieve homeostasis. If testing is conducted on a different day than the first protocol, subjects will be asked if there have been any changes to their daily routine or health, and markers to be used for identification of limb orientation during image-processing will be put on the residual limb. The subject will then don the prosthesis, have his/her weight taken, and then sit for 5-10 minutes with the prosthesis on.

The subject will remove his/her prosthesis, and then the uniform stress chamber will be positioned on the residual limb. A vacuum will be pulled from the chamber so that the elastomeric sleeve expands and easily allows the limb to be positioned within the chamber. The distal cup will be adjusted so that it is within 1 cm of the distal residual limb when the proximal end of the chamber touches the thigh just above the patella. The chamber pressure will then be slowly increased to 0 kPa so that the sleeve contacts the residual limb. Care will be taken to ensure the chamber is aligned with the limb axis and does not put undue pressure on the proximal thigh area.

Pressure application will then begin. The chamber pressure will slowly be increased to at least 13.8 kPa, a magnitude that has been found appropriate to achieve a reasonable thermal response, but not to cause discomfort to the subject. If the pressure is deemed by the prosthetist or subject to be excessive, then it will be reduced. Dynamic pressure application will be considered if static pressure proves to be ineffective in achieving a reasonable thermal response. Pressure application will continue for 5 minutes, an interval that has been shown effective in earlier studies to be comfortable to the subject and to induce an acceptable thermal response.

Immediately after pressure application is complete, IR image acquisition will be initiated and the pressure chamber removed. Quick release clamps on the top of the chamber will facilitate quick removal. The subject will then rest comfortably with the residual limb supported by the wheelchair pad while data are collected for 10 minutes.

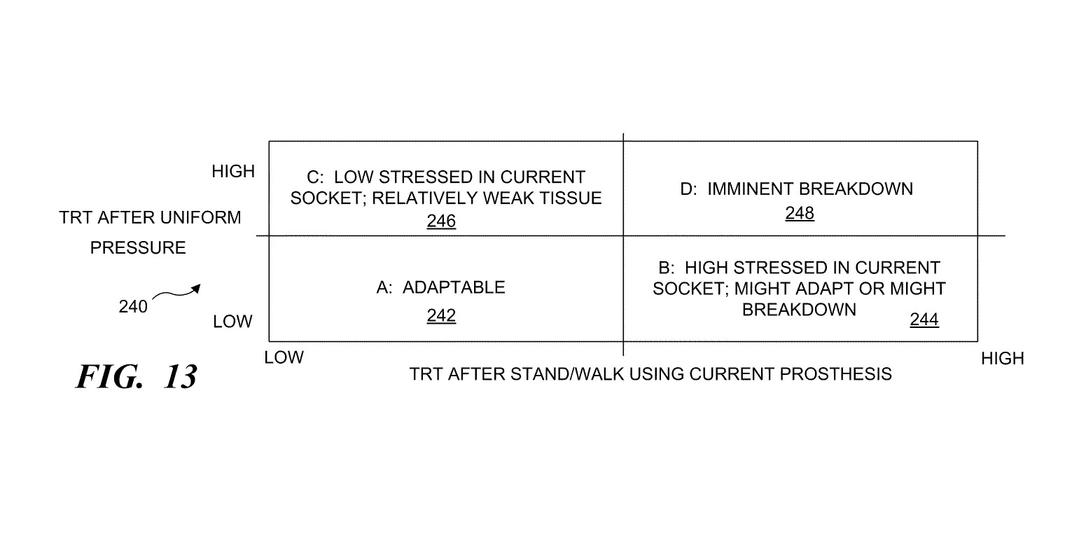

Data Analysis—Characterization of Local Tissue Response Based on TRT Data: A table 240 is shown in FIG. 13 that characterizes local tissue response after a subject has completed the standing/walk-in-place procedures described above. It is important to clarify that our definitions, i.e., the terms in table 240, are arbitrary definitions. If the hypothesized responses are proven by further studies, then they are appropriately named. The basis for these hypotheses is as follows: Sites that show short TRTs for the current socket under standing/walk-in-place conditions (A and C in blocks 242 and 246 of the table) are expected to be well-tolerating the applied interface stresses from the current socket. They might tolerate greater stress. It should be possible to determine if they are likely to tolerate greater stress by considering the uniform stress test TRT data. A short TRT result from uniform stress testing coupled with a short TRT from stand/walk-in-place (A) using the current prosthesis indicates a site well-tolerating a load that is not of extremely low magnitude during ambulation. This site would be expected tolerant to greater rectification, given that the tissue quality is so high compared to the rest of the residual limb. A long TRT result from uniform stress testing coupled with this short TRT from stand/walk-in-place (C) would indicate that, compared with the rest of the residual limb, interface stresses at that site are low. Such sites are untested and might turn into As after socket replacement or might turn into Ds. It is expected that rectification can be increased at these sites, or other means can be employed that elevate interface stress can be increased at these sites, but not appreciably, given that they need to adapt to become more load tolerant.

Sites that have long TRTs under standing/walk-in-place conditions (B and D in blocks 244 and 248 in the table) might be capable of adaptation or might be at risk of imminent breakdown. It is expected that it should be possible to distinguish between them by considering the uniform stress test TRT data. A long TRT under uniform stress coupled with a long TRT under standing/walk-in-place (D) suggests a site that is not well-tolerating stress in the current socket and is relatively weak compared to the rest of the residual limb. This site is at risk of imminent injury. A short TRT under uniform stress coupled with a long TRT under standing/walk-in-place (C) suggests a site that is relatively tolerant compared to the rest of the residual limb, but is not highly stressed in the current socket. It is expected that this site has not yet reached the “Y” in the road (see discussion of amputee subject data above) towards adaptation or breakdown. It would be risky to increase rectification at these sites, given this uncertainty.

The specification of “short” and “long” is arbitrary, but since this map is relative, this specification is acceptable. It is expected that characterization of regions in the upper 50 percentile as being long and those in the lower 50 percentile as short will be appropriate. However, testing of the hypotheses will determine, in part, if this is inadequate and a different percentile characterization is necessary.

During study of socket replacement processes, study researchers should take detailed notes from all clinical fitting sessions for the subjects conducted by the regular prosthetist, so as to ensure any socket changes are well documented. TRT data can be collected after standing/walk-in-place routines are carried out, and uniform pressure once a week after the socket is replaced, until no socket modifications are made for a 1-month period.

Exemplary Flowchart for Determining TRT Data from Collection of Images

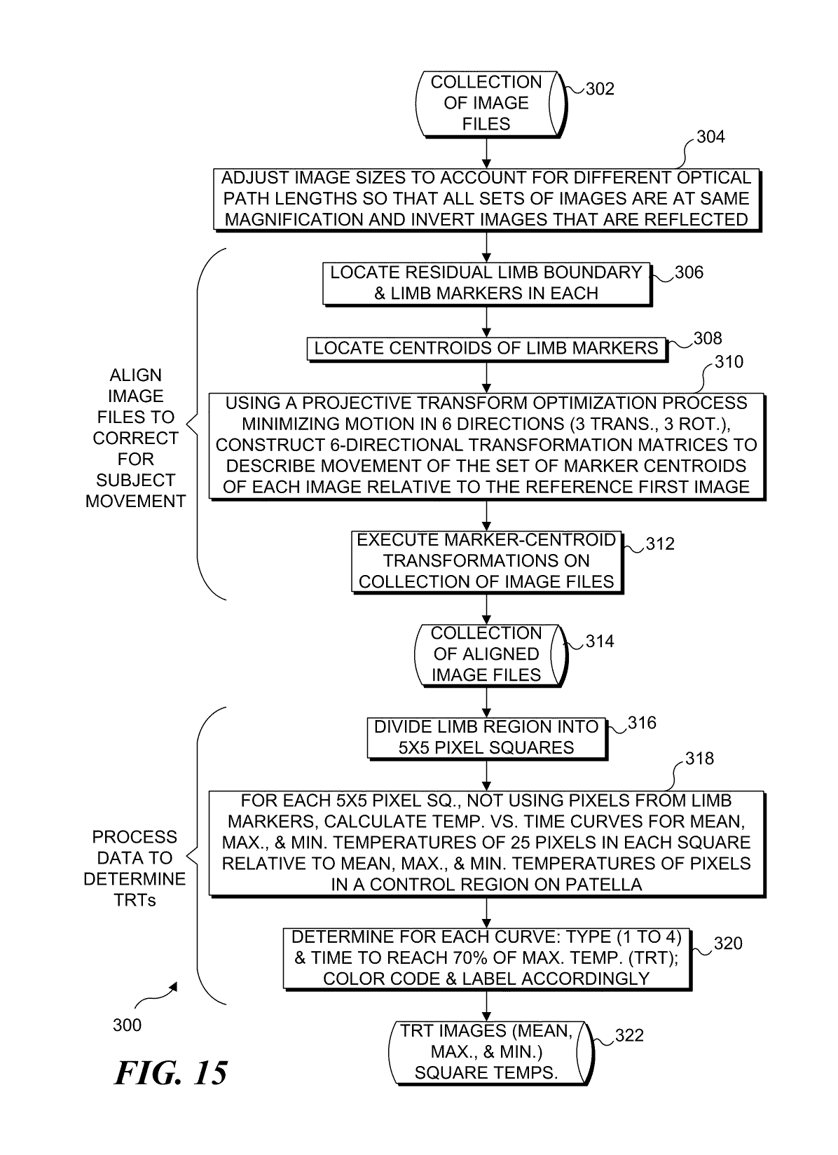

FIG. 15 is a flowchart 300 illustrating exemplary logical steps for processing a collection of image files 302 for a limb, to determine TRT image data for the limb. Accessing the collection of image files, a step 304 adjusts the image sizes to account for different optical path lengths so that all sets of the images for a limb are at the same magnification. Also, any images that are captured as a mirror reflection are inverted in this step. Steps 306, 308, 310, and 312 generally are implemented to align image files to correct for any movement by the subject between different images. In step 306, the residual limb boundary and the limb markers are located in each of the images produced by step 304. Step 308 provides for locating the centroids of the limb markers in those images. In step 310, using a projective transform optimization process, motion of the limb between the images is minimized in six directions, including along three translational orthogonal axes, and relative to rotations about the three axes. This process constructs six-directional transformation matrices to describe movement of the set of marker centroids of each image relative to a reference provided by the first image in the group. Step 312 then executes the marker-centroid transformation on the collections of image files for the limb, producing a collection of aligned image files 314.

Steps 316, 318, and 320 process the image data to determine the TRTs for each image. In step 316, the limb regions in each of the aligned images are divided into 5×5 pixel squares. For each 5×5 pixel square, and not using pixels from limb markers, step 318 calculates temperature vs. time for the 25 pixels in each square, relative to the mean, maximum, and minimum temperatures of pixels in a control region on the patella of the limb. More specifically, mean temperature in the 5×5 pixel square is subtracted from mean temperature at the control site; maximum temperature in the 5×5 pixel square is subtracted from maximum temperature at the control site; and, minimum temperature in the 5×5 pixel square is subtracted from minimum temperature at the control site. Step 320 then determines, for each curve, the type of curve (i.e., curves 1-4 as indicated in FIG. 4), and the time for the square to reach 70% of its maximum temperature, i.e., the TRT for the 5×5 pixel square. The squares are then color coded and labeled (with the curve type number 1-4), accordingly, in this step, yielding TRT images (along with the mean, maximum, and minimum temperatures for each 5×5 pixel square) 322.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for assessing a response of tissue to stress, comprising the steps of:
(a) applying stress to the tissue;
(b) immediately after the tissue is no longer stressed, collecting thermal images of the tissue over a time interval;
(c) processing the thermal images to determine temperature change data for the tissue over the time interval; and
(d) automatically evaluating the temperature change data over the time interval to characterize the response of the tissue to the stress that was applied.

2. The method of claim 1, further comprising the steps of:
(a) collecting control thermal images of a control tissue site that has not been stressed, wherein the control thermal images are collected over the time interval during which the thermal images of the tissue are collected;
(b) processing the control thermal images to determine control temperature data; and
(c) compensating the temperature change data for changes in the control temperature data that were not related to the response of the tissue to stress, including ambient conditions experienced both at the control tissue site and at the tissue that was stressed.

3. The method of claim 1, wherein the step of collecting thermal images comprises the step of collecting thermal images that include both thermal images produced in response to light received directly from the tissue, and thermal images produced in response to light from the tissue that has been reflected from a reflective surface.

4. The method of claim 1, further comprising the steps of:
(a) affixing a plurality of markers to the tissue before the step of applying the stress to the tissue;
(b) capturing visual images of the tissue over the time interval during the step of collecting the thermal images;
(c) using the plurality of markers for aligning the thermal images and the visual images, wherein the markers provide an indication of specific sites on the tissue.

5. The method of claim 1, wherein the step of automatically evaluating the temperature change data over the time interval comprises the steps of:
(a) dividing corresponding portions of the thermal images into a plurality of regions, wherein each region corresponds to a specific size pixel area;
(b) based upon the temperature change data, computing a thermal recovery time (TRT) for each region; and
(c) creating a visual map of the tissue indicating the TRT for each of the plurality of regions of the tissue, to visually show a rate of recovery of the tissue in each region after the stress is no longer being applied.

6. The method of claim 5, wherein the step of automatically evaluating the temperature change data over the time interval further comprises the steps of:
(a) based on the temperature change data over time for each region, identifying one of a plurality of different characteristic blood flow types for the tissue in the region; and
(b) indicating the characteristic blood flow type for the tissue in each region on the visual map.

7. The method of claim 5, wherein the step of automatically evaluating the temperature change data over the time interval further comprises the step of indicating a prospective condition of the tissue if periodically subjected to the stress that was applied.

8. The method of claim 1, wherein the tissue is on a residual limb of an amputee who uses a prosthetic socket that is worn on the residual limb, and wherein the step of applying the stress to the tissue comprises the step of causing the amputee to engage in a specific activity for a defined period of time while wearing the prosthetic socket on the residual limb, to assess an effect of the stress applied by the prosthetic socket on the tissue of the residual limb during the specific activity.

9. The method of claim 8, wherein the step of causing the amputee to engage in the specific activity comprises the step of causing the amputee to engage in at least one activity selected from the group of activities consisting of:
(a) standing while wearing the prosthetic socket on the residual limb; and
(b) walking-in-place while wearing the prosthetic socket on the residual limb.

10. The method of claim 8, further comprising the steps of applying a uniform stress to at least a portion of the tissue of the residual limb for a specific period of time, while the prosthetic socket is not being worn on the residual limb; and, then repeating steps (b)-(d) of claim 1.

11. The method of claim 10, further comprising the step of determining any difference in the response of the tissue to the stress that was applied as a result of wearing the prosthetic socket on the residual limb to the uniform stress that was applied to determine whether the response of the tissue is due to interface stress caused by the prosthetic socket, or due to tissue quality.

12. The method of claim 11, further comprising the step of categorizing the tissue of the residual limb in one of a plurality of different categories, based upon the response of the tissue on the residual limb to the uniform stress and to the stress caused by wearing the prosthetic socket on the residual limb.

13. The method of claim 12, wherein the step of categorizing comprises the step of determining whether a region of the tissue on the residual limb is in a category selected from the plurality of categories consisting of:
(a) adaptable, indicating that the tissue can adapt or is tolerant to the stress applied;
(b) highly stressed due to the stress applied by the prosthetic socket that is worn on the residual limb, so that the tissue might adapt or might breakdown;
(c) experiencing a low level of stress due to the prosthetic socket that is worn on the residual limb, but comprising relatively weak tissue; and
(d) at risk of imminent breakdown.

14. The method of claim 10, wherein the step of applying the uniform stress comprises one of the steps of:
(a) exposing the residual limb to a controlled pressure for the specific period of time; and
(b) rubbing the tissue of the residual limb with a mildly abrasive material for the specific period of time.

15. The method of claim 8, further comprising the step determining how to create a new prosthetic socket design for the residual limb as a function of the effect of the stress applied by the prosthetic socket on the tissue of the residual limb that was determined.

16. The method of claim 8, further comprising the step of selecting as a function of an effect of the stress applied by the prosthetic socket on the tissue of the residual limb, at least one of:
(a) prosthetic components; and
(b) settings for the prosthetic components.

17. A system for assessing a response of tissue to stress, comprising:
(a) a thermal imaging device that produces thermal images in response to infrared light and which is configurable to collect thermal images of tissue that has just been subjected to stress, over a time interval; and
(b) a computing device that is coupled to the thermal image device, to receive and store the thermal images, the computing device:
(i) processing the thermal images to determine temperature change data for the tissue over time; and
(ii) automatically evaluating the temperature change data over the time interval to characterize the response of the tissue to the stress that was applied.

18. The system of claim 17, further comprising at least one reflective surface that can be positioned to reflect infrared light traveling from the tissue, toward the thermal imaging device, so that thermal images include at least one image of the tissue from which the infrared light was reflected by the at least one reflective surface.

19. The system of claim 17, further comprising a digital camera for capturing conventional images of tissue over the time interval during which the thermal images are collected by the thermal imaging device.

20. The system of claim 19, further comprising markers that include an adhesive coating so that the markers can be removably applied to tissue before the thermal images of the tissue are collected by the thermal imaging device and the conventional images are captured by the digital camera.

21. The system of claim 20, wherein the computing device is programmed to use the markers that appear in the thermal images and in the conventional images to align the thermal images and the conventional images, alignment of said images compensating for any movement of the tissue during the time interval in which the thermal images were collected and the conventional images were captured.

22. The system of claim 17, wherein the computing device includes a display, and processes the thermal images by:
(a) dividing corresponding portions of the thermal images into a plurality of regions, wherein each region corresponds to a specific size pixel area;
(b) based upon the temperature change data, computing a thermal recovery time (TRT) for each region; and
(c) creating a visual map of the tissue indicating the TRT for each of the plurality of regions of the tissue, to visually present on the display, a rate of recovery of the tissue in each region after the stress is no longer being applied.

23. The system of claim 22, wherein the computing device identifies one of a plurality of different characteristic blood flow types for the tissue in each region, and indicates the characteristic blood flow type for the tissue in each region on the visual map presented on the display.

24. The system of claim 22, wherein the computing device further indicates a prospective condition of the tissue if periodically subjected to the stress that was applied before the thermal images were collected.

25. The system of claim 15, wherein the tissue is disposed on a residual limb of an amputee and the tissue on the residual limb is subjected to stress by a prosthetic socket that is worn on the residual limb during activity, further comprising a uniform stress chamber that is configured to receive the residual limb and to apply a uniform stress in the form of a controlled pressure applied to the tissue of the residual limb for a specific period of time, so that uniform stress thermal images of the tissue can immediately be collected over a specific time with the thermal imaging device after the residual limb is withdrawn from the uniform stress chamber, the computing device processing the uniform stress thermal images to determine uniform stress temperature change data for the tissue subjected to the uniform stress, and evaluating the uniform stress temperature change data collected over the specific time in comparison with the temperature change data taken after the stress was applied by the prosthetic socket, to determine if any difference in the response of the tissue is due to interface stress caused by the prosthetic socket, or due to tissue quality.

26. The system of claim 25, wherein the computing device categorizes the tissue of the residual limb in one of a plurality of different categories, based upon the response of the tissue on the residual limb to the uniform stress and to the stress caused by wearing the prosthetic socket on the residual limb.

27. The system of claim 26, wherein the computing device determines whether a region of the tissue on the residual limb is in a category selected from the plurality of categories consisting of:
(a) adaptable, indicating that the tissue can adapt or is tolerant to the stress applied;
(b) highly stressed due to the stress applied by the prosthetic socket that is worn on the residual limb, so that the tissue might adapt or might breakdown;
(c) experiencing a low level of stress due to the prosthetic socket that is worn on the residual limb, but comprising relatively weak tissue; and
(d) at risk of imminent breakdown.
